(19) United States
(12) Patent Application Publication
(10) Pub. No.: US 2025/0195796 A1
(43) Pub. Date: Jun. 19, 2025

(54) INHALATION DEVICE FOR ADMINISTRATION OF DRUGS BY INHALATION AND INHALATION SYSTEM

(71) Applicant: SIA EMTEKO HOLDING, RIGA (LV)

(72) Inventor: Mykola GUMENIUK, RIGA (LV)

(21) Appl. No.: 19/067,826

(22) Filed: Feb. 28, 2025

Related U.S. Application Data
(63) Continuation of application No. PCT/IB2023/058521, filed on Aug. 29, 2023.

(30) Foreign Application Priority Data
Aug. 30, 2022 (UA) A 2022 03158
Sep. 1, 2022 (UA) U 2022 03189
Sep. 1, 2022 (UA) U 2022 03190

(51) Int. Cl. A61M 11/00; A61M 15/00; A61M 16/00; A61M 16/10

(52) U.S. Cl. CPC A61M 11/005; A61M 15/0015; A61M 15/0018; A61M 15/0036; A61M 15/0085; A61M 15/0086; A61M 16/0093; A61M 16/109; A61M 2016/0024

(57) ABSTRACT

The disclosures is an inhalation device for administration of drugs by inhalation comprising the housing, the line of inhalation mixture formation fed to a person, the line of exhaled gas purification, and the control unit, all being positioned inside the housing. Additionally, provided an inhalation system comprising the inhalation device for administration of drugs by inhalation. The inhalation device for administration of drugs by inhalation and the inhalation system prevent the vapor of the drug from escaping into the environment, and ensured limited, clearly dosed and controlled delivery of the drug to a person.

### CROSS REFERENCE TO RELATED APPLICATION

This application is a continuation of International Patent Application No. PCT/IB2023/058521 with a filing date of Aug. 29, 2023, designating the United States, now pending, and further claims priority to Ukraine patent application a 2022 03158 with a filing date of Aug. 30, 2022, further claims priority to Ukraine patent application u 2022 03189 with a filing date of Sep. 1, 2022; further claims priority to Ukraine patent application u 2022 03190 with a filing date Sep. 1, 2022. The content of the aforementioned applications, including any intervening amendments thereto, are incorporated herein by reference.

### FIELD OF THE INVENTION

The disclosure relates to medical equipment, namely to devices for inhalation of volatile anesthetics.

### BACKGROUND OF THE INVENTION

The use of volatile liquids as active substances or volatile liquids containing active substances is well known. One such example is halogen-containing volatile liquids. Halogen-containing volatile liquids are described as useful for inducing and/or maintaining anesthesia (including amnesia, muscle paralysis and/or sedation) and/or analgesia, and, therefore, they may be useful as volatile anesthetics and/or volatile analgesics.

The easiest way for inducing anesthesia using volatile anesthetics is inhalation, i.e. inhalation of a vapor or aerosol particles of the volatile anesthetics by a person. A specific feature of anesthesia (analgesia) using volatile anesthetics is the need to control the dose of the volatile anesthetic inhaled by a person, since an overdose of the volatile anesthetic can cause a condition, such as general anesthesia.

In the international application WO 2005/118036 A2 (published on 15 Dec. 2005), an inhalation system comprising an oxygen or source of air supply, an aerosol chamber for a drug, a face mask and a filter, is described. The aerosol chamber for a drug is configured for the formation of an inhalation mixture by aerosolization of the drug in oxygen or air, and is configured to have an inlet for the oxygen or air supply, and an outlet for inhalation mixture. The source of oxygen or air is connected to the inlet for oxygen or air of the aerosol chamber for a drug with a tube. The outlet for inhalation mixture of the aerosol chamber for a drug is connected to the face mask with a tube containing a one-way valve that passes the inhalation mixture in the direction from the aerosol chamber for a drug to the face mask and does not pass it in the reverse direction. The filter is configured to purify of the drug the gas exhaled by a person, wherein the filter is configured to have an inlet for exhaled gas and an outlet for purified air. The inlet for exhaled gas of the filter is connected to the face mask with a tube containing a one-way valve that passes the exhaled gas in the direction from the face mask to the filter and does not pass the exhaled gas in the reverse direction.

The known inhalation system is used in the following way. A portion of the drug is poured into the aerosol chamber for a drug. Oxygen or air supply from the source of oxygen or air is turned on. Oxygen or air is constantly supplied to the aerosol chamber for a drug, where the inhalation mixture is formed. The inhalation mixture from the aerosol chamber for a drug enters the face mask. When inhaling, a person inhales the inhalation mixture, then, when exhaling, the exhaled gas is partially mixed with the inhalation mixture and enters the filter, where the drug is to be purified.

In medical technology, the aerosol chamber for a drug together with other devices necessary for the formation of the inhalation mixture are often called inhalation devices. The inhalation devices according to the modes of administering the inhalation mixture to the mouth or nose of a person are divided into active devices, where the administration of the inhalation mixture is carried out due to the constant administration of air or another gas, and passive devices, where the administration of the inhalation mixture is carried out only due to the flow of the air when a person inhales.

In the known inhalation system, the inhalation device of the active type is used. The disadvantage of such inhalation device is the constant inhalation of the drug, i.e., the administration of the drug is unlimited and uncontrolled, which, in the case of using a volatile anesthetic as a drug, for example, methoxyflurane, for pain relief, may lead to an overdose of the drug and the development of a condition during inhalation, such as general anesthesia, which is undesirable.

Another disadvantage of the known inhalation system is the design of the aerosol chamber for a drug, into which the drug must be poured. In the case of using a volatile anesthetic as a drug, when the volatile anesthetic is poured, a certain amount of vapor of the volatile anesthetic enters the air of the room where the patient is, and these vapors are inhaled by other people.

The objective technical problem of this disclosure is to develop an improved inhalation device that will be more economical, accurate and safe to use, as well as general-purpose for compatible use together with any delivery device and/or as part of the inhalation system.

Another objective technical problem of this disclosure is to develop an improved inhalation system that is safe, convenient and general-purpose for use, and enables achieving a more economical and accurate dosage of the drug due to the inclusion of an improved inhalation device.

Another objective technical problem of this disclosure is to expand the range and assortment means for volatile anesthetic inhalation, in particular, means for methoxyflurane inhalation, such as inhalation devices intended for use as part of the inhalation system, and the inhalation systems.

### SUMMARY OF THE INVENTION

The first objective technical problem is solved by a first disclosure being an inhalation device for the administration of drugs by inhalation (1) comprising a housing (4), a line of inhalation mixture formation administered to a person, a line of exhaled gas purification, a control unit (5) located in the housing (4), wherein the line of inhalation mixture formation administered to a person comprises an aerosol chamber (6), an inspiratory sensor (7), a port (8) configured for connecting a bottle containing the drug, a batcher (9), wherein the line of exhaled gas purification comprises a filter (10) configured to purify of the drug the gas exhaled by a person, wherein the aerosol chamber (6) comprises a housing (12) and a piezoelectric transducer (13) positioned in the housing (12) of the aerosol chamber and configured for the formation of an aerosol of the drug, wherein the housing (12) of the aerosol chamber (6) comprises an inlet for air supply (14), a hole for drug supply (15) and an outlet for inhalation mixture (16), wherein the inspiratory sensor (7) comprises an inlet for air (17) and an outlet for air (18), wherein the port (8) comprises an outlet for drug (19), wherein the batcher (9) is configured to administer the drug from the port (8) to the piezoelectric transducer (13), and comprises an inlet for drug (20) and an outlet for drug (21), wherein the filter (10) comprises an inlet (22), through which exhaled gas enters, and comprises an outlet for purified air (23), wherein the outlet for air (18) of the inspiratory sensor (7) is connected to the inlet for air supply (14) of the housing (12) of the aerosol chamber (6), wherein the outlet for drug (19) of the port (8) is connected to the inlet for drug (20) of the batcher (9), wherein the outlet for drug (21) of the batcher (9) passes through the hole for drug supply (15) of the housing (12) of the aerosol chamber (6), wherein the inspiratory sensor (7) has an electrical connection with the control unit (5), the piezoelectric transducer (13) has an electrical connection with the control unit (5), the batcher (9) has an electrical connection with the control unit (5).

In one aspect of the first disclosure, the aerosol chamber (6) can be configured in such a way that the housing (12) of the aerosol chamber (6) consists of an upper housing element (29) and a lower housing element (30), and the upper housing element (29) comprises a main part (31) in the form of a hollow cylinder open from below with a cylindrical side wall (32) and an upper end wall (33), wherein the upper end wall (33) comprises an outlet for inhalation mixture (16) in the form of a nozzle, and consists of a first side part (34) and a second side part (35) connected to the lower portion of the cylindrical side wall (32) so that they are located on opposite sides of the cylindrical side wall (32), wherein the first side part (34) comprises an inlet for air supply (14) in the form of a nozzle, the second side part (35) comprises a deepening (36), through the inner space of which air passes from the inner space of the lower housing element (30) to the inner space of the main part (31), and the upper side of the second side part (35) comprises a hole for drug supply (15), the lower housing element (30) comprises a longitudinal deepening (37), through the inner space of which air passes from the inlet for air supply (14) to the interior space of the deepening (36), wherein the aerosol chamber (6) further comprises a partition wall (38) positioned between the upper housing element (29) and the lower housing element (30) separating the inner space of the longitudinal deepening (37) from the inner space of the main part (31), and the portion of the partition wall (38) positioned under the inlet for air supply (14) comprises one or more holes (55), through which air passes from the inlet for air supply (14) to the inner space of the longitudinal deepening (37), and the portion of the partition wall (38) positioned under the hole for drug supply (15) comprises a hole (56), above or below which a piezoelectric transducer (13) is positioned.

In one aspect of the first disclosure, the aerosol chamber (6) further may comprise an air inlet valve (57) configured to introduce air into the aerosol chamber (6) through the inlet for air supply (14) and to prevent air from escaping from the aerosol chamber (6) through the inlet for air supply (14), and positioned in the housing (12) of the aerosol chamber (6) in the inlet for air supply (14) or near the inlet for air supply (14).

In one aspect of the first disclosure, the port (8) further can comprise the first needle (39) configured to have a through channel and to pierce the stopper on the bottle containing the drug and to administer the drug from the bottle containing the drug to the outlet for drug (19) of the port (8).

In one aspect of the first disclosure, the port (8) further can comprise an inlet for air (40), a second needle (41) configured to have a through channel and to pierce the stopper on the bottle containing the drug and administer the air from the inlet for air (40) to the bottle containing the drug.

In one aspect of the first disclosure, the inspiratory sensor (7) is configured to provide a signal to the control unit (5) during inhalation by a person, and the control unit (5) is configured to supply the power to the batcher (9) in order for the batcher (9) to deliver a dose of the drug to the aerosol chamber (6) only when receiving a signal from the inspiratory sensor (7) during inhalation by a person.

In one aspect of the first disclosure, the line of inhalation mixture formation administered to a person may comprise an element for forming an airflow (11) configured to force the formation of the air flow in the line of inhalation mixture formation and/or in the line of exhaled gas purification, and configured to comprise an inlet for air (42) and an outlet for air (43), wherein the element for forming an airflow (11) has an electrical connection with the control unit (5), an outlet for air (43) of the element for forming an airflow (11) is connected to the inlet for air (17) of the inspiratory sensor (7).

In one aspect of the first disclosure, the line of inhalation mixture formation administered to a person may comprise an element for forming an airflow (11) configured to force the formation of the air flow in the line of inhalation mixture formation and/or in the line of exhaled gas purification, and configured to comprise an inlet for air (42) and an outlet for air (43), and the element for forming an airflow (11) has an electrical connection with the control unit (5), an inlet for air (42) of the element for forming an airflow (11) is connected to the outlet for air (18) of the inspiratory sensor (7), and the outlet for air (43) of the element for forming an airflow (11) is connected to the inlet for air supply (14) of the housing (12) of the aerosol chamber (6).

In one aspect of the first disclosure, the line of exhaled gas purification may comprise an element for forming an airflow (11) configured to force the formation of the air flow in the line of inhalation mixture formation and/or in the line of exhaled gas purification, and configured to comprise an inlet for air (42) and an outlet for air (43), wherein the element for forming an airflow (11) has an electrical connection with the control unit (5), an inlet for air (42) of the element for forming an airflow (11) is connected to the outlet for purified air (23) of the filter (10).

In one aspect of the first disclosure, a fan can be used as an element for forming an airflow (11).

In one aspect of the first disclosure, the inhalation device for administration of drugs by inhalation (1) further may comprise at least one heating element configured to heat the housing (12) of the aerosol chamber (6) and configured to have an electrical connection with control unit (5).

In one aspect of the first disclosure, the aerosol chamber (6) further may comprise at least one heating element configured to heat the housing (12) of the aerosol chamber (6) and configured to have an electrical connection with control unit (5).

In one aspect of the first disclosure, the aerosol chamber (6) further may comprise at least one heating element configured to heat the air or inhalation mixture inside the housing (12) of the aerosol chamber (6) and configured to have an electrical connection with control unit (5).

In one aspect of the first disclosure, the drug is methoxyflurane.

The second objective technical problem is solved with a second disclosure being an inhalation system (3) comprising inhalation device for administration of drugs by inhalation (1), a delivery device (2) configured to deliver an inhalation mixture from the inhalation device for administration of drugs by inhalation (1) into the mouth or nose of a person during inhalation by a person and to remove the exhaled gas from the mouth or nose of the person during exhalation into the inhalation device for administration of drugs by inhalation (1), a flexible tube (24) and a flexible tube (25), wherein the inhalation device for administration of drugs by inhalation (1) comprises a housing (4), a line of inhalation mixture formation administered to a person, a line of exhaled gas purification, a control unit (5) located in the housing (4), wherein the line of inhalation mixture formation administered to a person comprises an aerosol chamber (6), an inspiratory sensor (7), a port (8) configured for connecting a bottle containing the drug, a batcher (9), wherein the line of exhaled gas purification comprises a filter (10) configured to purify of the drug the gas exhaled by a person, wherein the aerosol chamber (6) comprises a housing (12) and a piezoelectric transducer (13) positioned in the housing (12) of the aerosol chamber, and configured for the formation of an aerosol of the drug, wherein the housing (12) of the aerosol chamber (6) comprises an inlet for air supply (14), a hole for drug supply (15) and an outlet for inhalation mixture (16), wherein the inspiratory sensor (7) comprises an inlet for air (17) and an outlet for air (18), wherein the port (8) comprises an outlet for drug (19), wherein the batcher (9) is configured to administer the drug from the port (8) to the piezoelectric transducer (13), and comprises an inlet for drug (20) and an outlet for drug (21), wherein the filter (10) comprises an inlet (22), through which exhaled gas enters, and comprises an outlet for purified air (23), wherein the outlet for air (18) of the inspiratory sensor (7) is connected to the inlet for air supply (14) of the housing (12) of the aerosol chamber (6), wherein the outlet for drug (19) of the port (8) is connected to the inlet for drug (20) of the batcher (9), wherein the outlet for drug (21) of the batcher (9) passes through the hole for drug supply (15) of the housing (12) of the aerosol chamber (6), wherein the inspiratory sensor (7) has an electrical connection with the control unit (5), the piezoelectric transducer (13) has an electrical connection with the control unit (5), the batcher (9) has an electrical connection with the control unit (5), wherein the delivery device (2) comprises an inlet for inhalation mixture (26), an outlet for exhaled gas (27) and a breath inlet (28), through which, during inhalation by a person, the inhalation mixture is administered to the mouth or nose of a person, and, during exhalation by a person, exhaled gas is removed from the mouth or nose of a person, wherein the first end of the flexible tube (24) is connected to the outlet for inhalation mixture (16) of the housing (12) of the aerosol chamber (6), the second end of the flexible tube (24) is connected to the inlet for inhalation mixture (26) of the delivery device (2), wherein the first end of the flexible tube (25) is connected to the inlet (22) of the filter (10), the second end of the flexible tube (25) is connected to the outlet for exhaled gas (27) of the delivery device (2).

In one aspect of the second disclosure, the aerosol chamber (6) can be configured in such a way that the housing (12) of the aerosol chamber (6) consists of an upper housing element (29) and a lower housing element (30), and the upper housing element (29) comprises a main part (31) in the form of a hollow cylinder open from below with a cylindrical side wall (32) and an upper end wall (33), wherein the upper end wall (33) comprises an outlet for inhalation mixture (16) in the form of a nozzle, and consists of a first side part (34) and a second side part (35) connected to the lower portion of the cylindrical side wall (32) so that they are located on opposite sides of the cylindrical side wall (32), wherein the first side part (34) comprises an inlet for air supply (14) in the form of a nozzle, the second side part (35) comprises a deepening (36), through the inner space of which air passes from the inner space of the lower housing element (30) to the inner space of the main part (31), and the upper side of the second side part (35) comprises a hole for drug supply (15), the lower housing element (30) comprises a longitudinal deepening (37), through the inner space of which air passes from the inlet for air supply (14) to the interior space of the deepening (36), wherein the aerosol chamber (6) further comprises a partition wall (38) positioned between the upper housing element (29) and the lower housing element (30) separating the inner space of the longitudinal deepening (37) from the inner space of the main part (31), and the portion of the partition wall (38) positioned under the inlet for air supply (14) comprises one or more holes (55), through which air passes from the inlet for air supply (14) to the inner space of the longitudinal deepening (37), and the portion of the partition wall (38) positioned under the hole for drug supply (15) comprises a hole (56), above or below which a piezoelectric transducer (13) is positioned.

In one aspect of the second disclosure, the aerosol chamber (6) may contain an air inlet valve (57) configured to introduce air into the aerosol chamber (6) through the inlet for air supply (14) and prevent air from escaping from of the aerosol chamber (6) through the inlet for air supply (14), and positioned in the housing (12) of the aerosol chamber (6) in the inlet for air supply (14) or near the inlet for air supply (14).

In one aspect of the second disclosure, the delivery device (2) can comprise an inhalation channel through which the inhalation mixture administered to a person passes, an inlet for inhalation mixture (26) connected with the first end of the inhalation channel, the exhalation channel through which the exhaled gas is removed, the outlet for exhaled gas (27) connected to the first end of the exhalation channel, the exhalation valve positioned in the inhalation channel and open only during inhalation by a person, an exhalation valve positioned in the exhalation channel and open only during exhalation by a person, a breath chamber configured to administer an inhalation mixture from the inhalation channel to the mouth or nose of a person during inhalation by a person and to remove exhaled gas from the mouth or nose of a person into the exhalation channel during exhalation by a person, wherein the breath chamber comprises a first inlet connected to the second end of the inhalation channel, a second inlet connected to the second end of the exhalation channel, a breath inlet (28), through which, during inhalation by a person, an inhalation mixture is administered to the mouth or nose of a person, and during exhalation by a person, exhaled gas is removed from the mouth or nose of a person.

In one aspect of the second disclosure, the delivery device (2) can comprise a housing (44) comprising a first housing element (45) and a second housing element (46), a channel element (47) positioned in the housing (44), the breath chamber (48) positioned in the housing (44), wherein the channel element (47) comprises an inhalation channel (49) of a tubular shape, an exhalation channel (50) of a tubular shape, an inlet for inhalation mixture (26) configured in the form of a nozzle and connected to the first end of the inhalation channel (49), an outlet for exhaled gas (27) configured in the shape of a nozzle and connected to the first end of the exhalation channel (50), an inhalation valve (51) positioned inside the inhalation channel (49) and configured to be open only during inhalation by a person, an exhalation valve (52) positioned inside the exhalation channel (50) and configured to be open only during exhalation by a person, the breath chamber (48) comprises a first inlet (53) connected to the second end of the inhalation channel (49), a second inlet (54) connected to the second end of the exhalation channel (50), the breath inlet (28), through which the inhalation mixture is administered to the mouth or nose of a person during inhalation by a person and exhaled gas is removed from the mouth or nose of a person during exhalation by a person.

In one aspect of the second disclosure, the port (8) further can comprise the first needle (39) configured to have a through channel and to pierce the stopper on the bottle containing the drug and to administer the drug from the bottle containing the drug to the outlet for drug (19) of the port (8).

In one aspect of the second disclosure, the port (8) further can comprise an inlet for air (40), a second needle (41) configured to have a through channel and to pierce the stopper on the bottle containing the drug and to administer the air from the inlet for air (40) to the bottle containing the drug.

In one aspect of the second disclosure, the inspiratory sensor (7) is configured to provide a signal to the control unit (5) during inhalation by a person, and the control unit (5) is configured to supply the power to the batcher (9) in order for the batcher (9) to deliver a dose of the drug to the aerosol chamber (6) only when receiving a signal from the inspiratory sensor (7) during inhalation by a person.

In one aspect of the second disclosure, the inhalation system (3) further can comprise a plug configured to close the breath inlet (28) of the delivery device (2), and the line of inhalation mixture formation administered to a person further may comprise an element for forming an airflow (11) configured to force the formation of the air flow in the line of inhalation mixture formation and/or in the line of exhaled gas purification, and configured to comprise an inlet for air (42) and an outlet for air (43), wherein the element for forming an airflow (11) has an electrical connection with the control unit (5), an outlet for air (43) of the element for forming an airflow (11) is connected to the inlet for air (17) of the inspiratory sensor (7).

In one aspect of the second disclosure, the inhalation system (3) further can comprise a plug configured to close the breath inlet (28) of the delivery device (2), and the line of inhalation mixture formation administered to a person further may comprise an element for forming an airflow (11) configured to force the formation of the air flow in the line of inhalation mixture formation and/or in the line of exhaled gas purification, and configured to comprise an inlet for air (42) and an outlet for air (43), wherein the element for forming an airflow (11) has an electrical connection with the control unit (5), an inlet for air (42) of the element for forming an airflow (11) connected to outlet for air (18) of the inspiratory sensor (7), and an outlet for air (43) of the element for forming an airflow (11) is connected to the inlet for air supply (14) of the housing (12) of the aerosol chamber (6).

In one aspect of the second disclosure, the inhalation system (3) further can comprise a plug configured to close the breath inlet (28) of the delivery device (2), and the line of exhaled gas purification further can comprise an element for forming an airflow (11) configured to force the formation of the air flow in the line of inhalation mixture formation and/or in the line of exhaled gas purification, and configured to comprise an inlet for air (42) and an outlet for air (43), wherein the element for forming an airflow (11) has an electrical connection with the control unit (5), an inlet for air (42) of the element for forming an airflow (11) is connected to the outlet for purified air (23) of the filter (10).

In one aspect of the second disclosure, a fan can be used as an element for forming an airflow (11).

In one aspect of the second disclosure, the inhalation device for administration of drugs by inhalation (1) further may comprise at least one heating element configured to heat the housing (12) of the aerosol chamber (6) and configured to have an electrical connection with control unit (5).

In one aspect of the second disclosure, the aerosol chamber (6) further may comprise at least one heating element configured to heat the housing (12) of the aerosol chamber (6) and configured to have an electrical connection with control unit (5).

In one aspect of the second disclosure, the aerosol chamber (6) further may comprise at least one heating element configured to heat the air or inhalation mixture inside the housing (12) of the aerosol chamber (6) and configured to have an electrical connection with control unit (5).

In one aspect of the second disclosure, a flexible tube (24) may comprise at least one heating element configured to heat the flexible tube (24) or inhalation mixture that passes through the flexible tube (24) and configured to have an electrical connection with control unit (5).

In one aspect of the second disclosure, a flexible tube (25) may comprise at least one heating element configured to heat the flexible tube (25) or exhaled gas that passes through the flexible tube (25) and configured to have an electrical connection with control unit (5).

In one aspect of the second disclosure, the inhalation system (3) may comprise a mouthpiece, or a biteblock, or a face mask, each of which is configured to be connected to the breath inlet (28) of the delivery device (2).

In one aspect of the second disclosure, the drug is methoxyflurane.

### BRIEF DESCRIPTION OF THE DRAWINGS

Hereinafter, to better understand the essence of the disclosure, the possible examples of the embodiments of the claimed disclosure are shown using drawings in FIGS. 1-20.

FIG. 1 is a general view of the inhalation device for administration of drugs by inhalation (1).

FIG. 2 is a general view of the inhalation system (3).

FIG. 3 is a general view of the inhalation system (3) with the mouthpiece mounted on the delivery device (2).

FIG. 4 is a general view of the aerosol chamber (6) together with the batcher (9).

FIG. 5 is a view of the disassembled aerosol chamber (6) together with the batcher (9) according to one of the aspects.

FIG. 6 is a view of the disassembled aerosol chamber (6) together with the batcher (9) according to one of the aspects.

FIG. 7 is a sectional front view of the aerosol chamber (6) together with the batcher (9).

FIG. 8 is a diagram of air movement in the aerosol chamber (6).

FIG. 9 is a general view of the delivery device (2) together with the flexible tube (24) and the flexible tube (25).

FIG. 10 is a side view of the delivery device (2) together with the flexible tube (24) and the flexible tube (25).

FIG. 11 is a view of the disassembled delivery device (2).

FIG. 12 is a section A-A from FIG. 12.

FIG. 13 is a general view of the port (8) with the protective cap for the bottle containing the drug.

FIG. 14 is a sectional front view of the port (8) with the bottle containing the drug.

FIG. 15 is a front sectional view of the port (8) positioned inside the housing (4) with the bottle containing the drug.

FIG. 16 is a diagram of the inhalation device for administration of drugs by inhalation (1) according to one of the aspects.

FIG. 17 is a diagram of the inhalation system (3).

FIG. 18 is a diagram of the inhalation device for administration of drugs by inhalation (1) according to one of the aspects.

FIG. 19 is a diagram of the inhalation device for administration of drugs by inhalation (1) according to one of the aspects.

FIG. 20 is a diagram of the inhalation device for administration of drugs by inhalation (1) according to one of the aspects.

### DETAILED DESCRIPTION OF THE INVENTION

The first disclosure is the inhalation device for administration of drugs by inhalation (1).

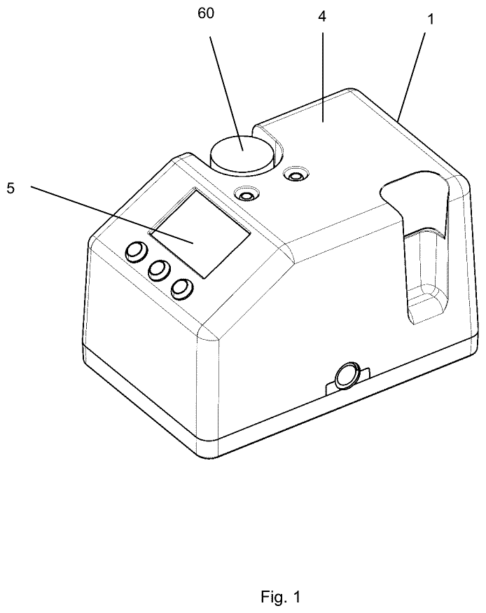

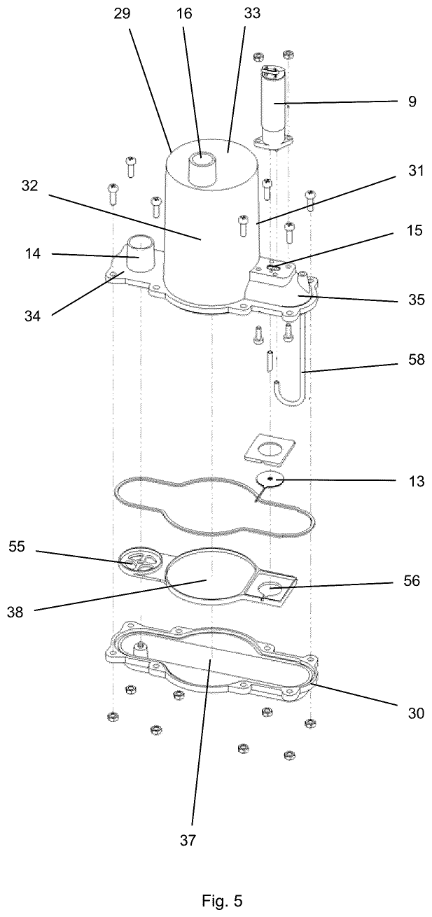

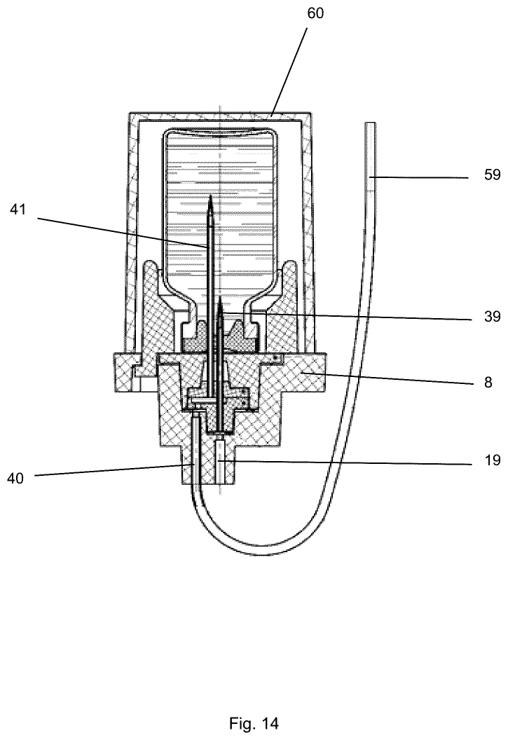

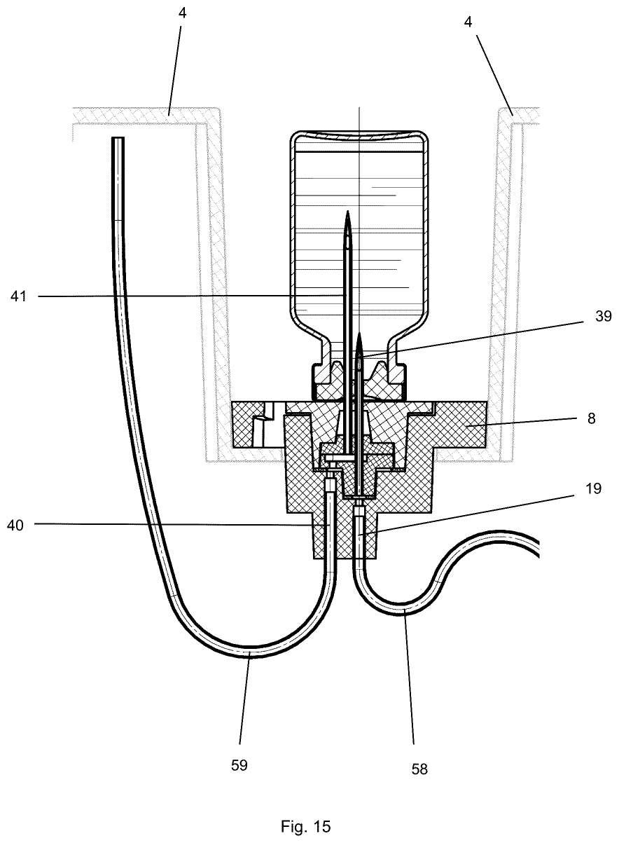

Different embodiments of the inhalation device for administration of drugs by inhalation (1) and structural elements of the inhalation device for administration of drugs by inhalation (1) are shown in FIGS. 1, 4-8, 13-16, and 18-20.

The second disclosure is the inhalation system (3).

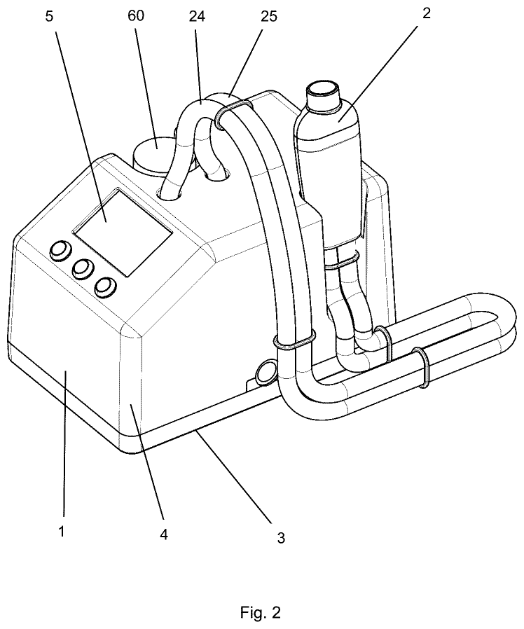

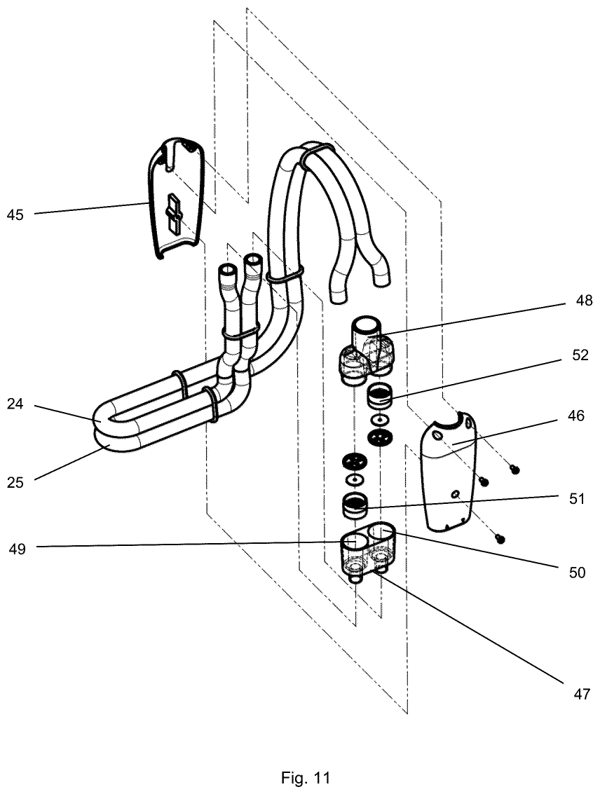

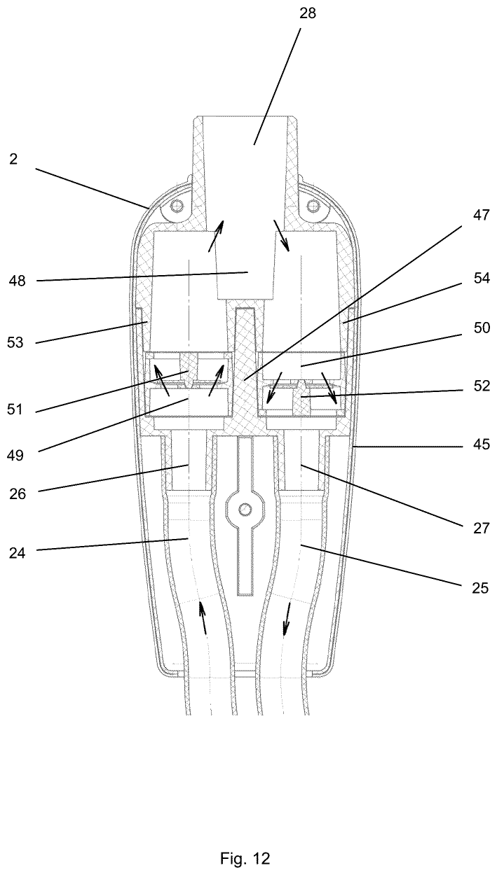

Different embodiments of the inhalation system (3) and structural elements of the inhalation system (3) are shown in FIGS. 2, 3, 9-12, and 17.

Since the basic element of the inhalation system (3), that ensures the functioning of the inhalation system (3), is the inhalation device for the administration of drugs by inhalation (1), it is apparent that various embodiments of the inhalation device for the administration of drugs by inhalation (1) result in different respective configurations of the inhalation system (3).

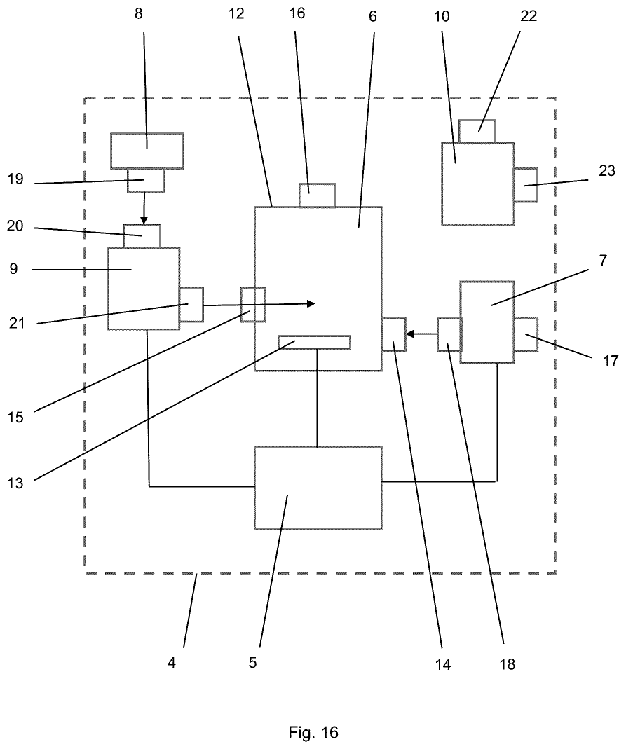

The inhalation device for administration of drugs by inhalation (1) according to the main embodiment of the first disclosure (the general view is shown in FIG. 1, the diagram is shown in FIG. 16) comprises the housing (4), the line of inhalation mixture formation administered to a person, the line of exhaled gas purification, and the control unit (5), all positioned inside the housing (4).

The line of exhaled gas purification comprises the filter (10) configured to purify of the drug the gas exhaled by a person, wherein the filter (10) comprises an inlet (22) through which exhaled gas enters, and an outlet for purified air (23). The filter (10) comprises a filter material that absorbs the drug from the gas exhaled by a person. As an example, the filter (10) may contain activated carbon, cartridges with activated carbon, for example, Delta Plus cartridges with activated carbon, as a filter material.

The line of inhalation mixture formation administered to a person consists of the structural elements, such as the aerosol chamber (6), the inspiratory sensor (7), the port (8) configured for connecting the bottle containing the drug, the batcher (9) that administers the drug from the port (8) to the aerosol chamber (6).

The aerosol chamber (6) comprises the housing (12) and the piezoelectric transducer (13) positioned inside the housing (12) of the aerosol chamber. The housing (12) of the aerosol chamber (6) comprises the inlet for air supply (14), the hole for drug supply (15), and the outlet for inhalation mixture (16).

The air enters the interior space of the aerosol chamber (6) through the inlet for air supply (14). The drug enters the interior space of the aerosol chamber (6) through the hole for drug supply (15). The inhalation mixture formed as a result of mixing the air and the aerosol of the drug leaves the aerosol chamber (6) through the outlet for inhalation mixture (16).

a. The piezoelectric transducer (13) is configured for the formation of the aerosol from a liquid drug. The piezoelectric transducer (13) means any item, in which alternating current is converted into high-frequency oscillations, and when liquid comes into contact with the item, aerosol particles of liquid are formed. An example of the piezoelectric transducer (13), that can be used in the first disclosure, is an ultrasonic membrane that is often used in state-of-the-art inhalation devices. The piezoelectric transducer (13) has an electrical connection with the control unit (5): the control unit (5) supplies alternating current to the piezoelectric transducer (13).

The port (8) is configured to connect the bottle containing the drug and to administer the drug from the bottle containing the drug to the inhalation system (3). The port (8) is configured to have the outlet for drug (19). Any item and/or device adapted for connecting bottles and having the outlet for supplying liquid from the bottle can be used as a port (8).

The batcher (9) is configured to administer the drug from the port (8) onto the piezoelectric transducer (13) of the aerosol chamber (6). The batcher (9) comprises the inlet for drug (20) and the outlet for drug (21). The inlet for drug (20) of the batcher (9) is connected to the outlet for drug (19) of the port (8), and the outlet for drug (21) of the batcher (9) passes through the hole for drug supply (15) of the housing (12) of the aerosol chamber (6) so that the drug that leaves through the outlet for drug (21) gets onto the piezoelectric transducer (13). Hereinafter, when it is stated that one structural element is connected to another structural element, it is understood that the connection can be either direct or through intermediate elements, for example, such as pipelines, through which a medium, such as air or drug, passes.

Any item, device, and/or system that can supply liquid can be used as the batcher (9). As an example, it can be solenoid pumps, which are often used in the inhalation devices and similar devices. The outlet for drug (21) means any tube connected to the batcher (9) and through which the drug can exit the batcher (9). The batcher (9) has an electrical connection with the control unit (5): the control unit (5) supplies the batcher (9) with the power supply that is necessary for the functioning of the batcher (9).

The inspiratory sensor (7) comprises the inlet for air (17) and the outlet for air (18). The outlet for air (18) of the inspiratory sensor (7) is connected to the inlet for air supply (14) of the housing (12) of the aerosol chamber (6). Any sensor that emits a signal when the air flow passes through it can be used as the inspiratory sensor. In this case, the air flow is formed in the sensor at the moment when a person inhales. That is why this structural element is called the inspiratory sensor. The inspiratory sensor (7) has an electrical connection with the control unit (5): the control unit (5) receives the signal emitted by the inspiratory sensor (7) during inhalation by a person, and, based on the signals from the inspiratory sensor (7), conducts specific control of the delivery of the drug to the aerosol chamber (6).

In the preferred embodiment of the first disclosure, the control unit (5) is configured to supply power to the batcher (9) in order for the batcher (9) to deliver a specific predetermined dose of the drug to the aerosol chamber (6) only when receiving a signal from the inspiratory sensor (7) during inhalation by a person. Due to this configuration of the inspiratory sensor (7) and the control unit (5), and the adjustment of the dose of the drug delivered during each administration of the drug to the aerosol chamber (6), the formation of the inhalation mixture inhaled by a person is performed only during the inhalation by a person, and at the same time, the amount of the drug administered to a person is limited and controlled. Respectively, this mode of operation of the inhalation device for administration of drugs by inhalation (1) results in clearly dosed and controlled administration of the drug to the human body, which, in the case of the use of the drugs, such as volatile anesthetics, enables preventing an overdose of the volatile anesthetics and preventing the development of a condition during inhalation, such as general anesthesia.

The volume of the drug dose administered onto the piezoelectric transducer (13) of the aerosol chamber (6) can be adjusted in various ways, for example, by adjusting the volumetric speed of the batcher (9) (for example, mechanical adjustment of the batcher (9)), as well as by changing the time of operation of the batcher (9) during inhalation by a person using the control unit (5) (for example, so that the drug is supplied by the batcher (9) all the time from the beginning of the inhalation by a person, or it is administered only for the first 1-2 seconds from the beginning of the inhalation by a person).

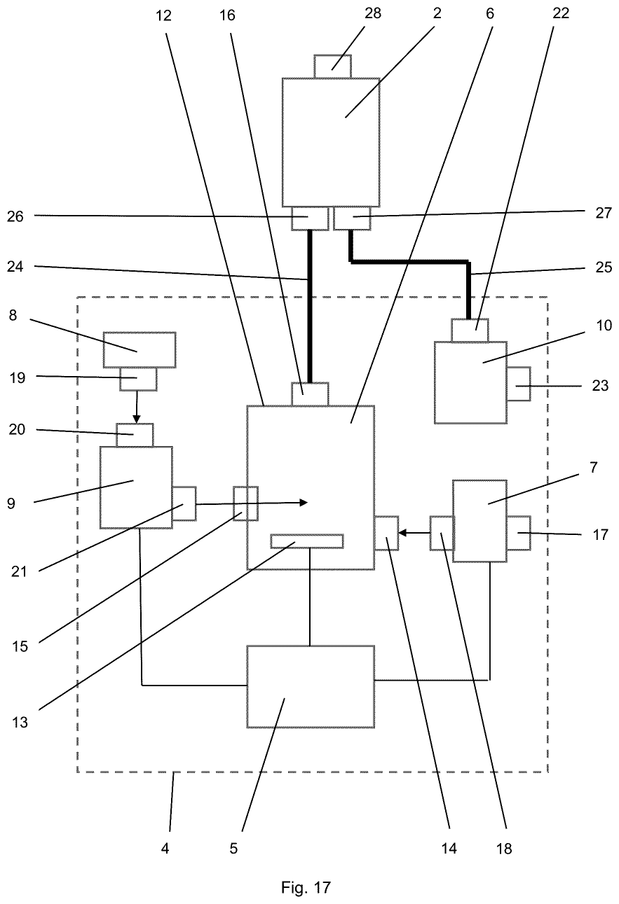

The inhalation system (3) according to the main embodiment of the second disclosure (the general view is shown in FIG. 1, FIG. 2, the diagram is shown in FIG. 17) comprises the inhalation device for administration of drugs by inhalation (1) according to the main embodiment of the first disclosure described above, and comprises the delivery device (2) configured to deliver the inhalation mixture from the inhalation device for administration of drug by inhalation (1) during inhalation by a person into the mouth or nose of a person and to remove the exhaled gas from the mouth or nose of a person to the inhalation device for administration of drug by inhalation (1) during exhalation by a person, the flexible tube (24) and the flexible tube (25).

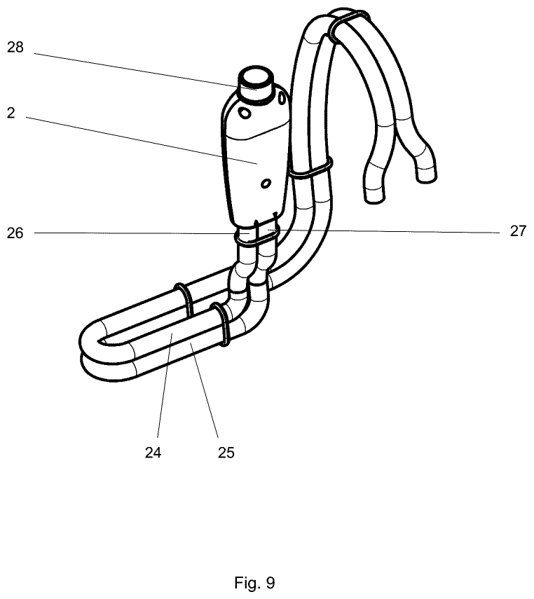

The delivery device (2) (shown in FIG. 9) comprises the inlet for inhalation mixture (26), the outlet for exhaled gas (27) and the breath inlet (28) through which the inhalation mixture is administered to the mouth or nose of a person during inhalation by a person and is removed from the mouth or nose of a person during exhalation by a person.

The flexible tube (24) is configured to supply the inhalation mixture from the aerosol chamber (6) to the delivery device (2). The first end of the flexible tube (24) is connected to the outlet for inhalation mixture (16) of the housing (12) of the aerosol chamber (6), the second end of the flexible tube (24) is connected to the inlet for inhalation mixture (26) of the delivery device (2).

The flexible tube (25) is configured to administer gas exhaled by a person from the delivery device (2) to the filter (10). The first end of the flexible tube (25) is connected to the inlet (22) of the filter (10), the second end of the flexible tube (25) is connected to the outlet for exhaled gas (27) of the delivery device (2).

Mouthpieces, biteblocks, or face masks may be attached to the breath inlet (28) of the delivery device (2) for providing inhalation.

Hereinafter, an example of using the inhalation system (3) according to the main embodiment of the second disclosure is described.

The bottle containing the drug is inserted into the port (8). The mouthpiece is attached to the breath inlet (28) of the delivery device (2). The control unit (5) is turned on, and the control unit (5) supplies the alternating current to the piezoelectric transducer (13). A person inserts the mouthpiece into the mouth and inhales.

When a person inhales a flow of air is formed in the aerosol chamber (6) and in the inspiratory sensor (7), the air enters the inner space of the housing (12) of the aerosol chamber (6) through the outlet for air (18) of the inspiratory sensor (7) and through the inlet for air supply (14) in the housing (12) of the aerosol chamber (6). At the same time, the inspiratory sensor (7) generates a signal that enters the control unit (5). The control unit (5) supplies power to the batcher (9), and the batcher (9) starts to supply the drug from the port (8) to the piezoelectric transducer (13). The drug, getting onto the piezoelectric transducer (13), is atomized to the aerosol form, which is continuously mixed with the air flow passing through the internal space of the aerosol chamber (6), with the formation of the inhalation mixture. The inhalation mixture enters the delivery device (2) through the outlet for inhalation mixture (16) in the housing (12) of the aerosol chamber (6), through the flexible tube (24) and the inlet for inhalation mixture (26) of the delivery device (2). From the delivery device (2), the inhalation mixture enters the mouth of a person through the breath inlet (28) of the delivery device (2) and the mouthpiece.

During the exhalation by a person, which follows the inhalation by a person, the flow of air through the inhalation sensor (7) stops, the signal received by the control unit (5) disappears, and the control unit (5) stops supplying power to the batcher (9), and accordingly the batcher (9) stops supplying the drug from the port (8) to the piezoelectric transducer (13). At the same time, gas exhaled by a person through the mouthpiece and breath inlet (28) of the delivery device (2) enters the delivery device (2). From the delivery device (2), the exhaled gas enters the filter (10) through the outlet for exhaled gas (27) of the delivery device (2), through the flexible tube (25) and the inlet (22) of the filter (10). When a person exhales, exhaled gas passes through the filter (10), where it is being purified of the drug, and the purified air leaves the filter (10) through the outlet for purified air (23) to the environment.

In the following cycles of inhalation-exhalation by a person, the functioning of the inhalation system (3) is repeated as described above.

According to one of the embodiments of the first disclosure and the second disclosure, in order to ensure the one-way movement of the inhalation mixture when a person inhales, and the one-way movement of exhaled gas when a person exhales, the aerosol chamber (6) can contain the air inlet valve (57) configured to introduce air into the aerosol chamber (6) through the inlet for air supply (14) and prevent air from leaving the aerosol chamber (6) through the inlet for air supply (14), and located in the housing (12) of the aerosol chamber (6) in the inlet for air supply (14) or near the inlet for air supply (14). The presence of the air inlet valve in the housing (12) of the aerosol chamber (6) results in the air entering the internal space of the housing (12) of the aerosol chamber (6) through the inlet for air supply (14) only during inhalation by a person, and not moving in the opposite direction during exhalation by a person. Accordingly, when a person inhales, the inhalation mixture passes from the aerosol chamber (6) to the delivery device (2), and, when a person exhales, the inhalation mixture cannot move from the delivery device (2) to the aerosol chamber (6).

According to another embodiment of the second disclosure, in order to fully ensure the one-way movement of the inhalation mixture when a person inhales, and the one-way movement of the exhaled gas when a person exhales, the delivery device (2) can comprise the inhalation channel through which the inhalation mixture administered to a person passes, the inlet for inhalation mixture (26) connected with the first end of the inhalation channel, the exhalation channel through which the exhaled gas is removed, the outlet for exhaled gas (27) connected to the first end of the exhalation channel, the exhalation valve positioned in the inhalation channel and open only during inhalation by a person, the exhalation valve positioned in the exhalation channel and open only during exhalation by a person, the breath chamber configured to administer the inhalation mixture from the inhalation channel to the mouth or nose of a person during inhalation by a person and to remove exhaled gas from the mouth or nose of a person into the exhalation channel during exhalation by a person, wherein the breath chamber comprises the first inlet connected to the second end of the inhalation channel, the second inlet connected to the second end of the exhalation channel, the breath inlet (28), through which, during inhalation by a person, the inhalation mixture is administered to the mouth or nose of a person, and during exhalation by a person, exhaled gas is removed from the mouth or nose of a person. Configuration of the delivery device (2) with the inhalation valve that is open only when the person inhales and with the exhalation valve that is open only when the person exhales provides that:

a. only the inhalation mixture moves in the direction from the aerosol chamber (6) to the mouth or nose of a person during inhalation by a person through the delivery device (2);
only exhaled gas moves in the direction from the mouth or nose of a person to the filter (10) during exhalation by a person through the delivery device (2).

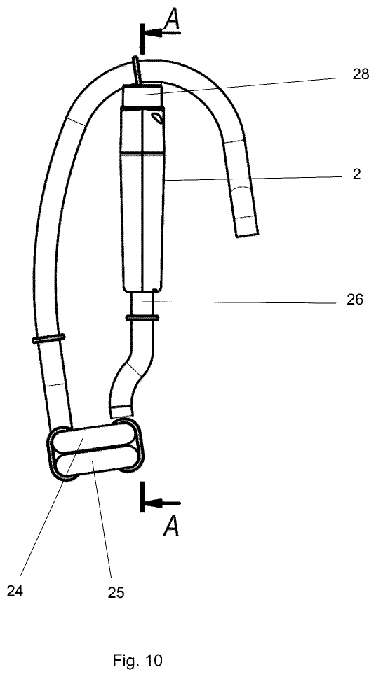

According to another embodiment of the second disclosure, in order to fully ensure the one-way movement of the inhalation mixture when a person inhales, and the one-way movement of exhaled gas when a person exhales, the delivery device (2) can comprise (shown in FIGS. 10, 11, and 12) the housing (44) that consists of the first housing element (45) and the second housing element (46).

The channel element (47) and the breath chamber (48) are positioned inside the housing (44).

The channel element (47) comprises:
the inhalation channel (49) of a tubular shape;
the exhalation channel (50) of a tubular shape;
the inlet for inhalation mixture (26) configured in the form of a nozzle and connected to the first end of the inhalation channel (49);
   a. the outlet for exhaled gas (27) configured in the form of a nozzle and connected to the first end of the exhalation channel (50);
   b. the inhalation valve (51) positioned inside the inhalation channel (49) and configured to be open only during inhalation by a person;
the exhalation valve (52) positioned inside the exhalation channel (50) and configured to be open only during exhalation by a person;

The breath chamber (48) comprises:
the first inlet (53) connected to the second end of the inhalation channel (49);
   a. the second inlet (54) connected to the second end of the exhalation channel (50);
the breath inlet (28) through which an inhalation mixture is administered to the mouth or nose of a person during inhalation by a person and exhaled gas is removed from the mouth or nose of a person during exhalation by a person.

The advantage of this embodiment of the second disclosure is the manufacturability of the delivery device (2), i.e., the simplicity of the manufacturing the elements of the delivery device (2) and simplicity of the assembly of the delivery device (2) from the manufactured elements. Configuration of the delivery device (2) according to this embodiment of the second disclosure ensures that:

a. only the inhalation mixture moves in the direction from the aerosol chamber (6) to the mouth or nose of a person during inhalation by a person through the delivery device (2);
only exhaled gas moves in the direction from the mouth or nose of a person to the filter (10) during exhalation by a person through the delivery device (2).

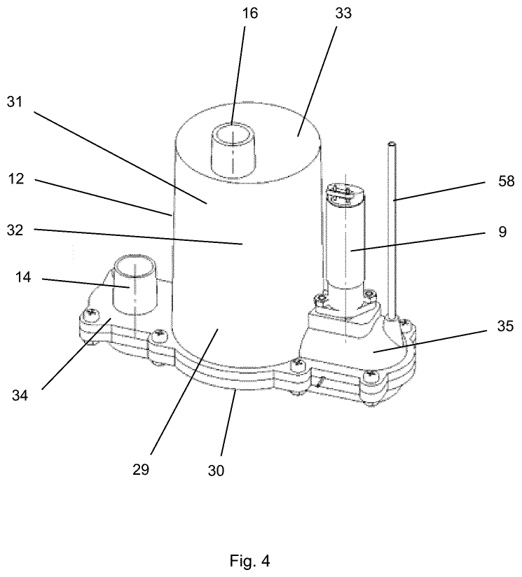

According to one of the embodiments of the first disclosure and the second disclosure, the aerosol chamber (6) can be configured in such a way (shown in FIGS. 4, 5, 6, 7, and 8 together with the batcher (9) attached to the housing (12)) that the housing (12) of the aerosol chamber (6) consists of the upper housing element (29) and the lower housing element (30), and the aerosol chamber (6) contains the partition wall (38) positioned between the upper housing element (29) and the lower housing element (30).

The upper housing element (29) comprises the main part (31), the first side part (34) and the second side part (35).

The main part (31) is configured in the form of a hollow cylinder open from below with the cylindrical side wall (32) and the upper end wall (33). The upper end wall (33) comprises the outlet for inhalation mixture (16) in the form of a nozzle.

The first side part (34) and the second side part (35) are connected to the lower portion of the cylindrical side wall (32) so that they are located on opposite sides of the cylindrical side wall (32) of the main part (31).

a. The first side part (34) comprises the inlet for air supply (14) in the form of a nozzle.

The second side part (35) comprises the deepening (36), through the inner space of which air passes from the inner space of the lower housing element (30) to the inner space of the main part (31). The upper side of the second side part (35) comprises the hole for drug supply (15).

a. The lower housing element (30) comprises the longitudinal deepening (37), through the inner space of which air passes from the inlet for air supply (14) to the inner space of the deepening (36) in the second side part (35).

The aerosol chamber (6) comprises the partition wall (38) positioned between the upper housing element (29) and the lower housing element (30) so that it separates the inner space of the longitudinal deepening (37) in the lower housing element (30) from the inner space of the main part (31). The portion of the partition wall (38) located under the inlet for air supply (14) comprises one or more holes (55), through which air passes from the inlet for air supply (14) to the inner space of the longitudinal deepening (37). The portion of the partition wall (38) located under the hole for drug supply (15) in the second side part (35) comprises the hole (56), above which or under which the piezoelectric transducer (13) is located.

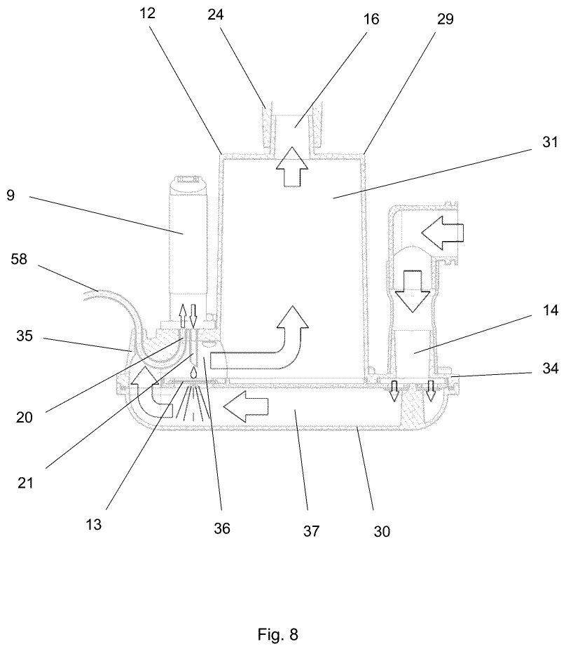

Due to the mutually opposite arrangement of the first side part (34) and the second side part (35), the presence of the partition wall (38), and the said configuration of the partition wall (38), and additionally due to separation of the inner space of the longitudinal deepening (37) in the lower housing element (30) only from the inner space of the main part (31) by the partition wall (38), and absence of the separation of the inner space of the longitudinal deepening (37) in the lower housing element (30) from the inner space of the deepening (36) in the second side part (35), the longitudinal deepening (37) in the lower housing element (30) becomes a channel, through the inner space of which air passes from the inlet for air supply (14) to the inner space of the deepening (36) in the second side part (35), and in which the liquid drug is atomized into the aerosol. The diagram of air movement in the aerosol chamber (6) according to this embodiment of the first disclosure and the second disclosure is shown in FIG. 8. In addition, the air in the inner space of the longitudinal deepening (37) in the lower housing element (30) moves in one direction, and the air containing the drug aerosol moves through the inner space of the deepening (36) in the second side part (35) to the inner space of the main of part (31) in the opposite direction, which results in the turbulence of the flow, enhances the mixing of the aerosol of the drug with the air, and, accordingly, results in the homogeneity of the inhalation mixture administered to a person during inhalation by a person.

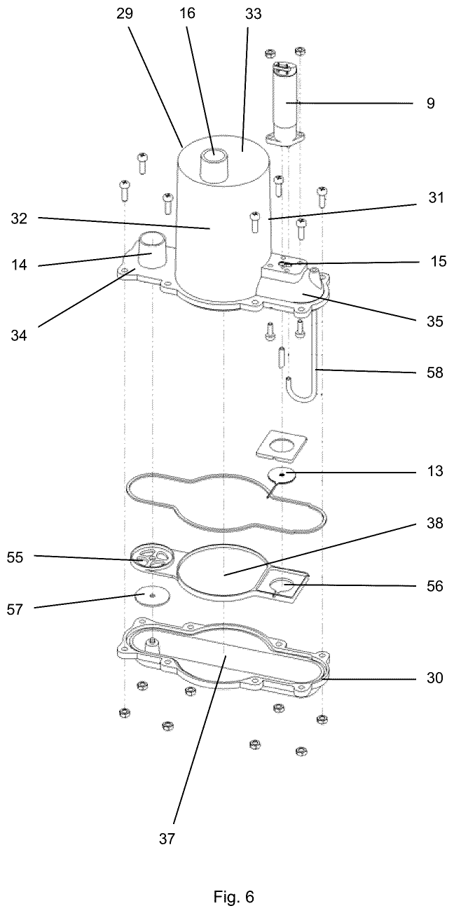

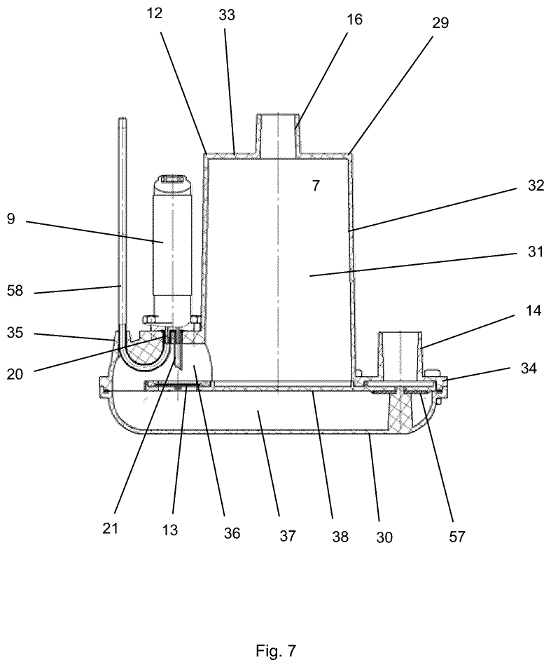

According to one of the embodiments of the aerosol chamber (6) described above, the aerosol chamber (6) may comprise the air inlet valve (57) (shown in FIGS. 6 and 7) that is located under one or more holes (55), and opens one or more holes (55) during inhalation by a person, and prevents air from leaving the aerosol chamber (6) through the inlet for air supply (14).

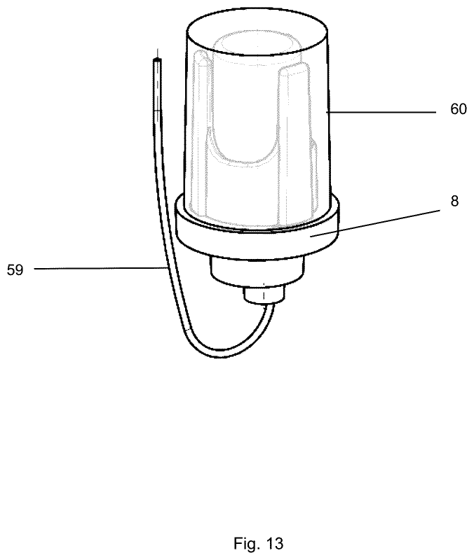

According to another embodiment, the port (8) can comprise (shown in FIGS. 13, 14, and 15) the outlet for drug (19) configured in the form of the hole, the first needle (39) configured to have the through channel and to pierce the stopper on the bottle containing the drug and to administer the drug from the bottle containing the drug to the outlet for drug (19). In addition, the port (8) may comprise the inlet for air (40) in the form of the hole, the second needle (41) configured to have the through channel and to pierce the stopper on the bottle containing the drug and to administer the air from the inlet for air (40) to the bottle containing the drug. The first needle (39) has a small length, preferably so that the upper end of the first needle (39) is close to the stopper on the bottle containing the drug. In this case, most of the liquid drug can come out of the bottle containing the drug. The second needle (41) is longer, preferably so that the upper end of the second needle (41) is close to the bottom of the bottle containing the drug. In this case, the level of the surface of the drug after inserting the bottle containing the drug into the port (8) will be lower than the upper end of the second needle (41), and air from the environment can freely enter the upper portion of the bottle containing the drug, while the pressure of the environment and the air in the bottle containing the drug will be equalized. To administer the drug from the port (8) to the batcher (9), the outlet for drug (19) is connected to the inlet for drug (20) of the batcher (9) with the flexible tube (58) inserted into the outlet for drug (19). To administer the air to the inlet for air (40), the flexible tube (59) is inserted into the hole of the inlet for air (40). The described embodiment of the port (8) enables using of standard bottles containing the drug closed with rubber stoppers in the claimed inhalation device for administration of drugs by inhalation (1) and the inhalation system (3). To protect the bottle containing the drug inserted into the port (8) from displacement or damage, the port (8) may have the protective cap (60) that is mounted on the port (8) and covers the bottle containing the drug from above.

According to another embodiment, the inhalation system (3) comprises the plug configured to close the breath inlet (28) of the delivery device (2), and the inhalation device for administration of drugs by inhalation (1) comprises the element for forming an airflow (11) configured to force the formation of the air flow in the line of inhalation mixture formation and/or in the line for exhaled gas purification. The element for forming an airflow (11) comprises the inlet for air (42) and the outlet for air (43), and has an electrical connection with the control unit (5). As the element for forming an airflow (11), any item and/or device capable of forming the air flow, such as a fan, can be used.

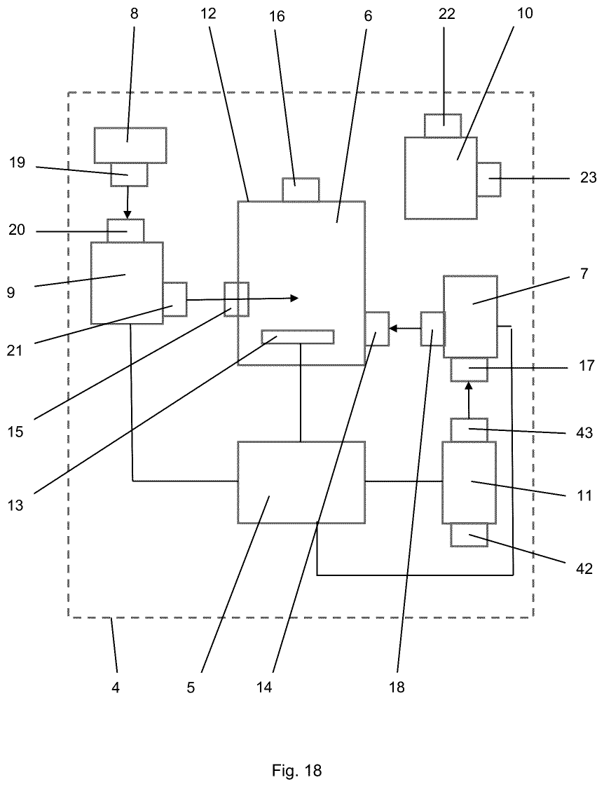

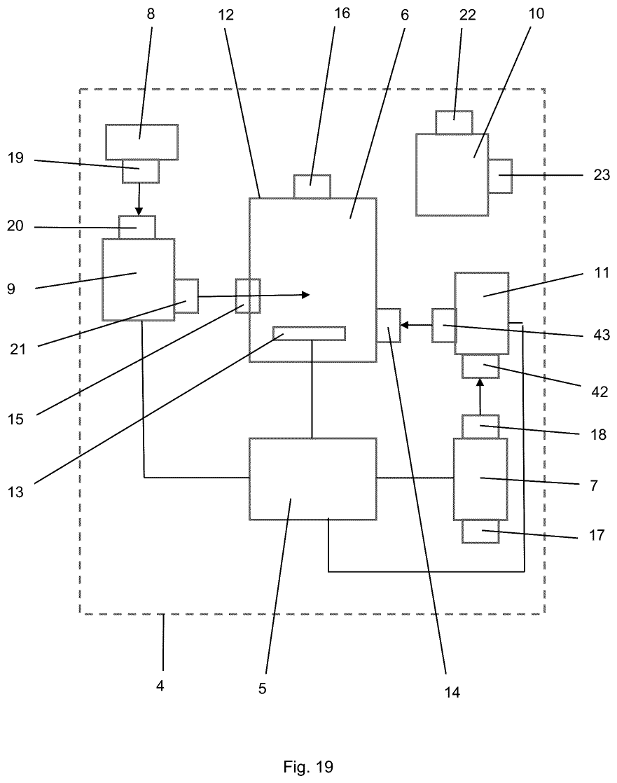

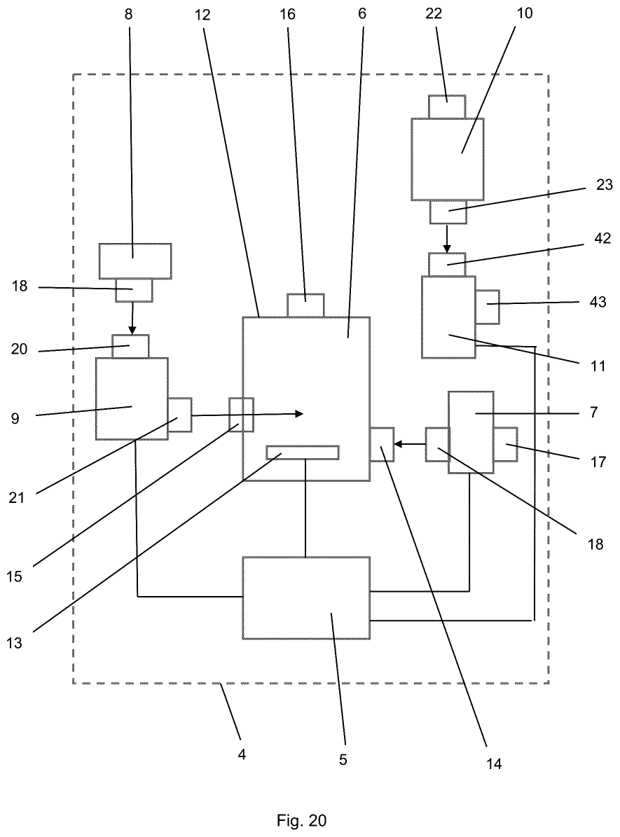

There are several embodiments, according to which the element for forming an airflow (11) can be installed in the inhalation device for administration of drugs by inhalation (1). According to one of the embodiments (shown in the diagram in FIG. 18), the element for forming an airflow (11) is installed in the line of inhalation mixture formation administered to a person, so that the outlet for air (43) of the element for forming an airflow (11) is connected to the inlet for air (17) of the inspiratory sensor (7). According to another embodiment (shown in the diagram in FIG. 19), the element for forming an air flow (11) is installed in the line of inhalation mixture formation administered to a person, so that the inlet for air (42) of the element for forming an air flow (11) is connected to the outlet for air (18) of the inspiratory sensor (7), and the outlet for air (43) of the element for forming an air flow (11) is connected to the inlet for air supply (14) of the housing (12) of the aerosol chamber (6). According to yet another embodiment (shown in the diagram in FIG. 20), the element for forming an air flow (11) is installed in the line for exhaled gas purification, so that the inlet for air (42) of the element for forming an air flow (11) is connected to the outlet for purified air (23) of the filter (10).

The presence of the element for forming an air flow (11) and the plug, which is to be put on to close the breath inlet (28) of the delivery device (2), enables blowdown of the inhalation system (3) of the drug residues. Such blowdown is necessary in the case when volatile anesthetic, for example, methoxyflurane, is used as a drug administered to a person.

The blowdown of the inhalation system (3) is conducted as follows. After using the inhalation system (3) for inhalation, the plug is put into the breath inlet (28) of the delivery device (2). Using the control unit (5), the system blowdown option is selected, after which the control unit supplies the element for forming an airflow (11) with power. After supplying power to the element for forming an airflow (11), the element for forming an airflow (11) creates a forced air flow that passes through the inspiratory sensor (7), the aerosol chamber (6), the flexible tube (24), the delivery device (2), the flexible tube (25), and filter (10). After a certain time, the control unit (5) stops supplying power to the element for forming an airflow (11), after which the plug is removed out of the breath inlet (28) of the delivery device (2), and the device is ready for further use.

The inhalation device for administration of drugs by inhalation (1), and, accordingly, the inhalation system (3), can be embodied both in a stationary mode with powering from a 220- or 110-volt network, and in a mobile mode with the possibility of powering from the elements of the power supply, such as accumulators or batteries. The mobile mode enables using the inhalation system (3) in various cases, for example, during emergency situations at the scene of events, when a person may be injured and needs anesthesia with a volatile anesthetic. Taking into account that in winter the temperature of the air is below zero degrees, anesthesia with the volatile anesthetic and administration of the air with such a low temperature will be problematic and ineffective, since the volatile anesthetic will condense in the aerosol chamber (6), the flexible tubes (24) and (25), and the device for delivery (2). To prevent this problem, the inhalation system (3) may comprise the heating elements.

In one embodiment, the inhalation device for administration of drugs by inhalation (1) may comprise at least one heating element configured to heat the housing (12) of the aerosol chamber (6) and configured to have an electrical connection with control unit (5). In this case, one or more heating elements, that externally heat the housing (12) of the aerosol chamber (6), are positioned in the housing (4).

In another embodiment, the aerosol chamber (6) may comprise at least one heating element configured to heat the housing (12) of the aerosol chamber (6) and configured to have an electrical connection with control unit (5). In this case, one or more heating elements, that directly heat the housing (12) of the aerosol chamber (6), are positioned in the housing (12) of the aerosol chamber (6).

In yet another embodiment, the aerosol chamber (6) may comprise at least one heating element configured to heat the air or the inhalation mixture in the housing (12) of the aerosol chamber (6) and configured to have an electrical connection with control unit (5). In this case, one or more heating elements, that directly heat the air or the inhalation mixture in the housing (12) of the aerosol chamber (6), are positioned in the housing (12) of the aerosol chamber (6).

In yet another embodiment, the flexible tube (24) comprises at least one heating element configured to heat the flexible tube (24) or to heat the inhalation mixture that passes through the flexible tube (24), and to have an electrical connection with the control unit (5). In this case, the flexible tube (24) has one or more heating elements that either directly heat the material of the flexible tube (24) or directly heat the inhalation mixture that passes through the flexible tube (24).

In yet another embodiment, the flexible tube (25) comprises at least one heating element configured to heat the flexible tube (25) or to heat the exhaled gas that passes through the flexible tube (25), and to have an electrical connection with the control unit (5). In this case, the flexible tube (25) has one or more heating elements inside that either directly heat the material of the flexible tube (25) or directly heat the exhaled gas that passes through the flexible tube (25).

It is apparent to one skilled in the art that it is possible to combine different embodiments of the heating element arrangements in different structural elements of the inhalation system (3).

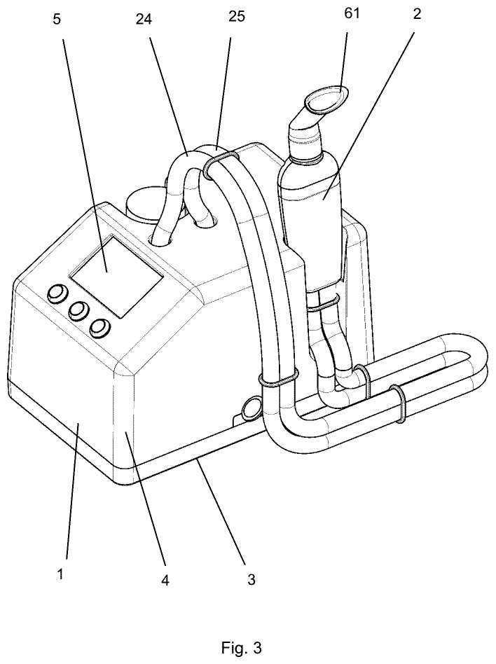

Since the convenience of the inhalation requires using the products that make administration of the inhalation mixture to the mouth or nose of a person convenient, for example, the mouthpiece, or the biteblock, or the face mask, the inhalation system (3) may comprise the mouthpiece or the face mask configured to be connected to the breath inlet (28) of the delivery device (2). An example of the embodiment of the inhalation system (3) with the mouthpiece attached to the breath inlet (28) of the delivery device (2) is shown in FIG. 3.

The technical contribution of the claimed inhalation device for administration of drugs by inhalation (1) and the inhalation system (3) resides in the following:
prevention of the drug vapors (for example, such as a volatile anesthetics) from entering the environment;
prevention of unnecessary losses of the drug at the stage of loading the drug into the inhalation device or the inhalation system, which enables achieving greater economy in terms of the amount of the drug;
greater safety for the patient, i.e. a precise and controlled dosage of the drug enables avoiding the overdose and undesirable side effects;
greater safety for people, i.e. since the drug does not escape into the environment, there is no risk of inhalation of the drug by people who are not targets and do not need it;
convenience and versatility in use, i.e. the inhalation device can be used together with any acceptable delivery device (the mouthpiece, or the biteblock, or the face mask), in particular, as part of the claimed inhalation system, which ensures the highest convenience and efficiency for a person, as well as various methods of the drug delivery (through the nose or mouth).

The given examples only illustrate the technical subject matter claimed, but do not limit them.

The disclosures relate to medical equipment, namely to devices for inhalation of volatile anesthetics.

Herein provided an inhalation device for administration of drugs by inhalation (1) comprising the housing (4), the line of inhalation mixture formation fed to a person, the line of exhaled gas purification, and the control unit (5), all being positioned inside the housing (4). Additionally, provided an inhalation system (3) comprising the inhalation device for administration of drugs by inhalation (1), the delivery device (2) configured to deliver the inhalation mixture from the inhalation device for administration of drug by inhalation (1) during inhalation by a person into the mouth or nose of a person and to remove the exhaled gas from the mouth or nose of a person to the inhalation device for administration of drug by inhalation (1) during exhalation by a person, the flexible tube (24), and the flexible tube (25).

The inhalation device for administration of drugs by inhalation (1) and the inhalation system (3) prevent the vapor of the drug from escaping into the environment, and ensured limited, clearly dosed and controlled delivery of the drug to a person.

I claim:

1. An inhalation device for the administration of drugs by inhalation characterized in that the inhalation device comprising a housing (4), a line of inhalation mixture formation administered to a person, a line of exhaled gas purification, a control unit (5) located in the housing (4), wherein the line of inhalation mixture formation administered to a person comprises an aerosol chamber (6), an inspiratory sensor (7), a port (8) configured for connecting a bottle containing the drug, a batcher (9), wherein the line of exhaled gas purification comprises a filter (10) configured to purify of the drug the gas exhaled by a person, wherein the aerosol chamber (6) comprises a housing (12) and a piezoelectric transducer (13) positioned in the housing (12) of the aerosol chamber and configured for the formation of an aerosol of the drug, wherein the housing (12) of the aerosol chamber (6) comprises an inlet for air supply (14), a hole for drug supply (15) and an outlet for inhalation mixture (16), wherein the inspiratory sensor (7) comprises an inlet for air (17) and an outlet for air (18), wherein the port (8) comprises an outlet for drug (19), wherein the batcher (9) is configured to administer the drug from the port (8) to the piezoelectric transducer (13), and comprises an inlet for drug (20) and an outlet for drug (21), wherein the filter (10) comprises an inlet (22), through which exhaled gas enters, and comprises an outlet for purified air (23), wherein the outlet for air (18) of the inspiratory sensor (7) is connected to the inlet for air supply (14) of the housing (12) of the aerosol chamber (6), wherein the outlet for drug (19) of the port (8) is connected to the inlet for drug (20) of the batcher (9), wherein the outlet for drug (21) of the batcher (9) passes through the hole for drug supply (15) of the housing (12) of the aerosol chamber (6), wherein the inspiratory sensor (7) has an electrical connection with the control unit (5), the piezoelectric transducer (13) has an electrical connection with the control unit (5), the batcher (9) has an electrical connection with the control unit (5).

2. An inhalation device according to claim 1, wherein the aerosol chamber (6) is configured in such a way that the housing (12) of the aerosol chamber (6) consists of an upper housing element (29) and a lower housing element (30), and the upper housing element (29) comprises a main part (31) in the form of a hollow cylinder open from below with a cylindrical side wall (32) and an upper end wall (33), wherein the upper end wall (33) comprises an outlet for inhalation mixture (16) in the form of a nozzle, consists of a first side part (34) and a second side part (35) connected to the lower portion of the cylindrical side wall (32) so that they are located on opposite sides of the cylindrical side wall (32), wherein the first side part (34) comprises an inlet for air supply (14) in the form of a nozzle, the second side part (35) comprises a deepening (36), through the inner space of which air passes from the inner space of the lower housing element (30) to the inner space of the main part (31), and the upper side of the second side part (35) comprises a hole for drug supply (15), the lower housing element (30) comprises a longitudinal deepening (37), through the inner space of which air passes from the inlet for air supply (14) to the interior space of the deepening (36), wherein the aerosol chamber (6) further comprises a partition wall (38) positioned between the upper housing element (29) and the lower housing element (30) separating the inner space of the longitudinal deepening (37) from the inner space of the main part (31), and the portion of the partition wall (38) positioned under the inlet for air supply (14) comprises one or more holes (55), through which air passes from the inlet for air supply (14) to the inner space of the longitudinal deepening (37), and the portion of the partition wall (38) positioned under the hole for drug supply (15) comprises a hole (56), above or below which a piezoelectric transducer (13) is positioned.

3. The inhalation device according to claim 1, wherein the aerosol chamber (6) further comprises an air inlet valve (57) configured to introduce air into the aerosol chamber (6) through the inlet for air supply (14) and to prevent air from escaping from of the aerosol chamber (6) through the inlet for air supply (14), and positioned in the housing (12) of the aerosol chamber (6) in the inlet for air supply (14) or near the inlet for air supply (14).

4. The inhalation device according to claim 1, wherein the port (8) further comprises the first needle (39) configured to have a through channel and to pierce the stopper on the bottle containing the drug and to administer the drug from the bottle containing the drug to the outlet for drug (19) of the port (8).

5. The inhalation device according to claim 4, wherein the port (8) further comprises inlet for air (40), and further comprises the second needle (41) configured to have a through channel and to pierce the stopper on the bottle containing the drug and to administer the air from the inlet for air (40) to the bottle containing the drug.

6. The inhalation device according to claim 1, wherein the inspiratory sensor (7) is configured to provide a signal to the control unit (5) during inhalation by a person, and the control unit (5) is configured to administer the power to the batcher (9) in order for the batcher (9) to deliver a dose of the drug to the aerosol chamber (6) only when receiving a signal from the inspiratory sensor (7) during inhalation by a person.

7. The inhalation device according to claim 1, wherein the line of inhalation mixture formation administered to a person further comprises an element for forming an airflow (11) configured to force the formation of the air flow in the line of inhalation mixture formation and/or in the line of exhaled gas purification, and configured to comprise an inlet for air (42) and an outlet for air (43), wherein the element for forming an airflow (11) has an electrical connection with the control unit (5), an outlet for air (43) of the element for forming an airflow (11) is connected to the inlet for air (17) of the inspiratory sensor (7).

8. The inhalation device according to claim 1, wherein the line of inhalation mixture formation administered to a person further comprises an element for forming an airflow (11) configured to force the formation of the air flow in the line of inhalation mixture formation and/or in the line of exhaled gas purification, and configured to comprise an inlet for air (42) and an outlet for air (43), and the element for forming an airflow (11) has an electrical connection with the control unit (5), an inlet for air (42) of the element for forming an airflow (11) is connected to the outlet for air (18) of the inspiratory sensor (7), and the outlet for air (43) of the element for forming an airflow (11) is connected to the inlet for air supply (14) of the housing (12) of the aerosol chamber (6).

9. The inhalation device according to claim 1, wherein the line of exhaled gas purification further comprises an element for forming an airflow (11) configured to force the formation of the air flow in the line of inhalation mixture formation and/or in the line of exhaled gas purification, and configured to comprise an inlet for air (42) and an outlet for air (43), wherein the element for forming an airflow (11) has an electrical connection with the control unit (5), an inlet for air (42) of the element for forming an airflow (11) is connected to the outlet for purified air (23) of the filter (10).

10. The inhalation device according to claim 7, wherein a fan is used as an element for forming an airflow (11).

11. The inhalation device according to claim 1, wherein further comprises at least one heating element configured to heat the housing (12) of the aerosol chamber (6) and configured to have an electrical connection with control unit (5).

12. The inhalation device according to claim 1, wherein aerosol chamber (6) further comprises at least one heating element configured to heat the housing (12) of the aerosol chamber (6) and configured to have an electrical connection with control unit (5).

13. The inhalation device according to claim 1, wherein aerosol chamber (6) further comprises at least one heating element configured to heat the air or the inhalation mixture in the housing (12) of the aerosol chamber (6) and configured to have an electrical connection with control unit (5).

14. The inhalation device according to claim 1, wherein the volatile anesthetic is methoxyflurane.

15. An inhalation system (3), wherein the inhalation system comprising inhalation device for administration of drugs by inhalation (1), a delivery device (2) configured to deliver an inhalation mixture from the inhalation device for administration of drugs by inhalation (1) into the mouth or nose of a person during inhalation by a person and remove the exhaled gas from the mouth or nose of the person during exhalation into the inhalation device for administration of drugs by inhalation (1), a flexible tube (24) and a flexible tube (25), wherein the inhalation device for administration of drugs by inhalation (1) comprises a housing (4), a line of inhalation mixture formation administered to a person, a line of exhaled gas purification, a control unit (5) located in the housing (4), wherein the line of inhalation mixture formation administered to a person comprises an aerosol chamber (6), an inspiratory sensor (7), a port (8) configured for connecting a bottle containing the drug, a batcher (9), wherein the line of exhaled gas purification comprises a filter (10) configured to purify of the drug the gas exhaled by a person, wherein the aerosol chamber (6) comprises a housing (12) and a piezoelectric transducer (13) positioned in the housing (12) of the aerosol chamber, and configured for the formation of an aerosol of the drug, wherein the housing (12) of the aerosol chamber (6) comprises an inlet for air supply (14), a hole for drug supply (15) and an outlet for inhalation mixture (16), wherein the inspiratory sensor (7) comprises an inlet for air (17) and an outlet for air (18), wherein the port (8) comprises an outlet for drug (19), wherein the batcher (9) is configured to administer the drug from the port (8) to the piezoelectric transducer (13), and comprises an inlet for drug (20) and an outlet for drug (21), wherein the filter (10) comprises an inlet (22), through which exhaled gas enters, and comprises an outlet for purified air (23), wherein the outlet for air (18) of the inspiratory sensor (7) is connected to the inlet for air supply (14) of the housing (12) of the aerosol chamber (6), wherein the outlet for drug (19) of the port (8) is connected to the inlet for drug (20) of the batcher (9), wherein the outlet for drug (21) of the batcher (9) passes through the hole for drug supply (15) of the housing (12) of the aerosol chamber (6), wherein the inspiratory sensor (7) has an electrical connection with the control unit (5), the piezoelectric transducer (13) has an electrical connection with the control unit (5), the batcher (9) has an electrical connection with the control unit (5), wherein the delivery device (2) comprises an inlet for inhalation mixture (26), an outlet for exhaled gas (27) and a breath inlet (28), through which, during inhalation by a person, the inhalation mixture is administered to the mouth or nose of a person, and, during exhalation by a person, exhaled gas is removed from the mouth or nose of a person, wherein the first end of the flexible tube (24) is connected to the outlet for inhalation mixture (16) of the housing (12) of the aerosol chamber (6), the second end of the flexible tube (24) is connected to the inlet for inhalation mixture (26) of the delivery device (2), wherein the first end of the flexible tube (25) is connected to the inlet (22) of the filter (10), the second end of the flexible tube (25) is connected to the outlet for exhaled gas (27) of the delivery device (2).

16. The inhalation system according to claim 15, wherein the aerosol chamber (6) is configured in such a way that the housing (12) of the aerosol chamber (6) consists of an upper housing element (29) and a lower housing element (30), and the upper housing element (29) comprises a main part (31) in the form of a hollow cylinder open from below with a cylindrical side wall (32) and an upper end wall (33), wherein the upper end wall (33) comprises an outlet for inhalation mixture (16) in the form of a nozzle, and consists of a first side part (34) and a second side part (35) connected to the lower portion of the cylindrical side wall (32) so that they are located on opposite sides of the cylindrical side wall (32), wherein the first side part (34) comprises an inlet for air supply (14) in the form of a nozzle, and the second side part (35) comprises a deepening (36), through the inner space of which air passes from the inner space of the lower housing element (30) to the inner space of the main part (31), and the upper side of the second side part (35) comprises a hole for drug supply (15), the lower housing element (30) comprises a longitudinal deepening (37), through the inner space of which air passes from the inlet for air supply (14) to the interior space of the deepening (36), wherein the aerosol chamber (6) further comprises a partition wall (38) positioned between the upper housing element (29) and the lower housing element (30) separating the inner space of the longitudinal deepening (37) from the inner space of the main part (31), and the portion of the partition wall (38) positioned under the inlet for air supply (14) comprises one or more holes (55), through which air passes from the inlet for air supply (14) to the inner space of the longitudinal deepening (37), and the portion of the partition wall (38) positioned under the hole for drug supply (15) comprises a hole (56), above or below which a piezoelectric transducer (13) is positioned.

17. The inhalation system according to claim 15, wherein the aerosol chamber (6) contains an air inlet valve (57) configured to introduce air into the aerosol chamber (6) through the inlet for air supply (14) and to prevent air from escaping from the aerosol chamber (6) through the inlet for air supply (14), and positioned in the housing (12) of the aerosol chamber (6) in the inlet for air supply (14) or near the inlet for air supply (14).

18. The inhalation system according to claim 15, wherein the delivery device (2) comprises an inhalation channel through which the inhalation mixture administered to a person passes, an inlet for inhalation mixture (26) connected with the first end of the inhalation channel, the exhalation channel through which the exhaled gas is removed, the outlet for exhaled gas (27) connected to the first end of the exhalation channel, the exhalation valve positioned in the inhalation channel and open only during inhalation by a person, an exhalation valve positioned in the exhalation channel and open only during exhalation by a person, a breath chamber configured to administer an inhalation mixture from the inhalation channel to the mouth or nose of a person during inhalation by a person and to remove exhaled gas from the mouth or nose of a person into the exhalation channel during exhalation by a person, wherein the breath chamber comprises a first inlet connected to the second end of the inhalation channel, a second inlet connected to the second end of the exhalation channel, a breath inlet (28), through which, during inhalation by a person, an inhalation mixture is administered to the mouth or nose of a person, and during exhalation by a person, exhaled gas is removed from the mouth or nose of a person.

19. The inhalation system according to claim 15, wherein the delivery device (2) comprises a housing (44) comprising a first housing element (45) and a second housing element (46), a channel element (47) positioned in the housing (44), the breath chamber (48) positioned in the housing (44), wherein the channel element (47) comprises an inhalation channel (49) of a tubular shape, an exhalation channel (50) of a tubular shape, an inlet for inhalation mixture (26) configured in the form of a nozzle and connected to the first end of the inhalation channel (49), an outlet for exhaled gas (27) configured in the shape of a nozzle and connected to the first end of the exhalation channel (50), an inhalation valve (51) positioned inside the inhalation channel (49) and configured to be open only during inhalation by a person, an exhalation valve (52) positioned inside the exhalation channel (50) and configured to be open only during exhalation by a person, the breath chamber (48) comprises a first inlet (53) connected to the second end of the inhalation channel (49), a second inlet (54) connected to the second end of the exhalation channel (50), the breath inlet (28), through which the inhalation mixture is administered to the mouth or nose of a person during inhalation by a person and exhaled gas is removed from the mouth or nose of a person during exhalation by a person.

20. The inhalation system according to claim 15, wherein the port (8) further comprises the first needle (39) configured to have a through channel and to pierce the stopper on the bottle containing the drug and to administer the drug from the bottle containing the drug to the outlet for drug (19) of the port (8).

21. The inhalation system according to claim 15, wherein the port (8) further comprises inlet for air (40), and further comprises the second needle (41) configured to have a through channel and to pierce the stopper on the bottle containing the drug and to administer the air from the inlet for air (40) to the bottle containing the drug.

22. The inhalation system according to claim 15, wherein the inspiratory sensor (7) is configured to provide a signal to the control unit (5) during inhalation by a person, and the control unit (5) is configured to administer the power to the batcher (9) in order for the batcher (9) to deliver a dose of the drug to the aerosol chamber (6) only when receiving a signal from the inspiratory sensor (7) during inhalation by a person.

23. The inhalation system according to claim 15, wherein further comprises a plug configured to close the breath inlet (28) of the delivery device (2), and the line of inhalation mixture formation administered to a person further comprises an element for forming an airflow (11) configured to force the formation of the air flow in the line of inhalation mixture formation and/or in the line of exhaled gas purification, and configured to comprise an inlet for air (42) and an outlet for air (43), wherein the element for forming an airflow (11) has an electrical connection with the control unit (5), an outlet for air (43) of the element for forming an airflow (11) is connected to the inlet for air (17) of the inspiratory sensor (7).

24. The inhalation system according to claim 15, wherein further comprises a plug configured to close the breath inlet (28) of the delivery device (2), and the line of inhalation mixture formation administered to a person further comprises an element for forming an airflow (11) configured to force the formation of the air flow in the line of inhalation mixture formation and/or in the line of exhaled gas purification, and configured to comprise an inlet for air (42) and an outlet for air (43), and the element for forming an airflow (11) has an electrical connection with the control unit (5), an inlet for air (42) of the element for forming an airflow (11) is connected to the outlet for air (18) of the inspiratory sensor (7), and the outlet for air (43) of the element for forming an airflow (11) is connected to the inlet for air supply (14) of the housing (12) of the aerosol chamber (6).

25. The inhalation system according to claim 15, wherein further comprises a plug configured to close the breath inlet (28) of the delivery device (2), and the line of exhaled gas purification further comprises an element for forming an airflow (11) configured to force the formation of the air flow in the line of inhalation mixture formation and/or in the line of exhaled gas purification, and configured to comprise an inlet for air (42) and an outlet for air (43), wherein the element for forming an airflow (11) has an electrical connection with the control unit (5), an inlet for air (42) of the element for forming an airflow (11) is connected to the outlet for purified air (23) of the filter (10).

26. The inhalation system according to claim 15, wherein a fan is used as an element for forming an airflow (11).

27. The inhalation system according to claim 15, wherein inhalation device for administration of drugs by inhalation (1) further comprises at least one heating element configured to heat the housing (12) of the aerosol chamber (6) and configured to have an electrical connection with control unit (5).

28. The inhalation system according to claim 15, wherein aerosol chamber (6) further comprises at least one heating element configured to heat the housing (12) of the aerosol chamber (6) and configured to have an electrical connection with control unit (5).

29. The inhalation system according to claim 15, wherein aerosol chamber (6) further comprises at least one heating element configured to heat the air or the inhalation mixture inside the housing (12) of the aerosol chamber (6) and configured to have an electrical connection with control unit (5).

30. The inhalation system according to claim 15, wherein the flexible tube (24) comprises at least one heating element configured to heat the flexible tube (24) or to heat the inhalation mixture that passes through the flexible tube (24), and configured to have an electrical connection with the control unit (5).

31. The inhalation system according to claim 15, wherein the flexible tube (25) comprises at least one heating element configured to heat the flexible tube (25) or to heat the exhaled gas that passes through the flexible tube (25), and configured to have an electrical connection with the control unit (5).

32. The inhalation system according to claim 15, wherein further comprises a mouthpiece, or a biteblock, or a face mask, each of which is configured to be connected to the breath inlet (28) of the delivery device (2).

33. The inhalation system according to claim 15, characterized in that the volatile anesthetic is methoxyflurane.
